(19) United States
(12) Patent Application Publication
(10) Pub. No.: US 2008/0139489 A1
(43) Pub. Date: Jun. 12, 2008

(54) USE OF KETOLIDES FOR PREVENTING ARTERIAL THROMBOTIC COMPLICATIONS RELATED TO ATHEROSCLEROSIS

(75) Inventors: Francis Petit, Colombes (FR); Francoise Vacheron, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Aventis Pharma S.A., Antony (FR)

(21) Appl. No.: 11/863,329

(22) Filed: Sep. 28, 2007

Related U.S. Application Data
(63) Continuation of application No. 09/554,772, filed on May 16, 2000, now abandoned.

(30) Foreign Application Priority Data
Nov. 17, 1997 (FR) 97/14358

(51) Int. Cl. A61K 31/7048; A61P 7/00

(52) U.S. Cl. 514/29

(57) ABSTRACT

The invention relates to a novel therapeutic application of ketolides. The invention relates to the use of ketolides for the preparation of pharmaceutical compositions intended for preventing arterial thrombotic complications associated with atherosclerosis.

The present invention relates to a novel therapeutic application of ketolides.

The invention relates to the use of ketolides and pharmaceutically acceptable salts thereof for the preparation of pharmaceutical compositions intended for preventing arterial thrombotic complications associated with atherosclerosis.

The term “ketolide” refers to erythromycin derivatives lacking cladinose in position 3. These products have antibiotic properties (Antimicrobial Agents and Chemotherapy 1997, vol. 41, pp. 2149 to 2158, or 1997 vol. 41, pp. 454 to 459 or Lettre de l'infectiologue 1997, vol. 12, pp. 46 to 54).

Ketolides are also described, for example, in European Patents 0487411, 596802, 606024, 614905, 676409, 680967 and 799833 and International Patent Application WO 98/25942.

Among the preferred ketolides of the invention, mention may be made of the compounds of the formula (I)

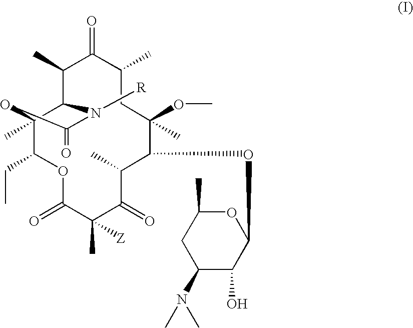

in which R represents a radical

(CH₂)ₘOₙ(X)YAr

in which
m represents the number 0 or 1,
n represents the number 0 or 1,
X represents a radical (NH)ₐ, CH₂ or SO₂ with a representing the number 0 or 1,
Y represents a radical (CH₂)_{b}—(CH═CH)_{c}—(CH₂)_{d} with c=0 or 1 and b+c+d≦8,
Z represents a hydrogen or halogen atom,
Ar represents an optionally substituted aryl or heteroaryl radical.

The aryl radical can be a phenyl or naphthyl radical.

The substituted or unsubstituted heterocyclic radical can be a thienyl, furyl, pyrrolyl, thiazolyl or oxazolyl radical, an imidazolyl radical, for example a 4-(3-pyridyl)-1H-imidazolyl radical, a thiadiazolyl, pyrazolyl or isopyrazolyl radical, a pyridyl, pyrimidyl, pyridazinyl or pyrazinyl radical, or alternatively an indolyl, benzofuryl, benzothiazyl or quinolyl radical.

These aryl radicals can contain one or more groups chosen from the group consisting of hydroxyl radicals, halogen atoms, NO₂ radicals, CN radicals, alkyl, alkenyl or alkynyl radicals, O-alkyl, O-alkenyl or O-alkynyl radicals, S-alkyl, S-alkenyl or S-alkynyl radicals and N-alkyl, N-alkenyl or N-alkynyl radicals, containing up to 12 carbon atoms optionally substituted by one or more halogen atoms, the radical

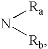

where Ra and Rb, which may be identical or different, represent a hydrogen atom or an alkyl radical containing up to 12 carbon atoms, the radical

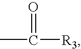

where R₃ represents an alkyl radical containing up to 12 carbon atoms, or an optionally substituted aryl or heteroaryl radical, where the aryl, O-aryl or S-aryl carboxylic or aryl, O-aryl or S-aryl heterocyclic 5- or 6-membered radicals comprise one or more heteroatoms, optionally substituted by one or more of the substituents mentioned below.

Preferred heterocycles which may be mentioned are, inter alia,

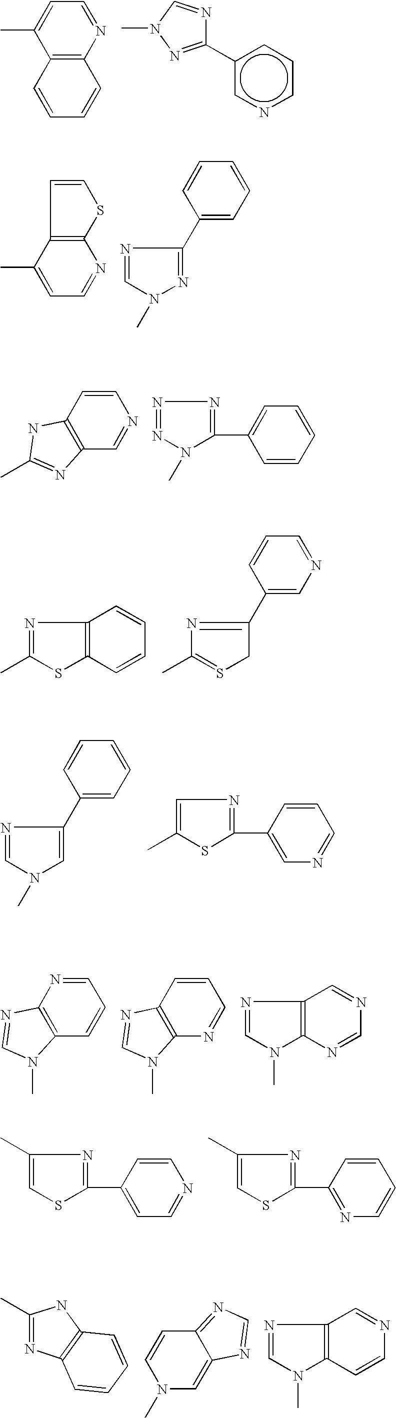

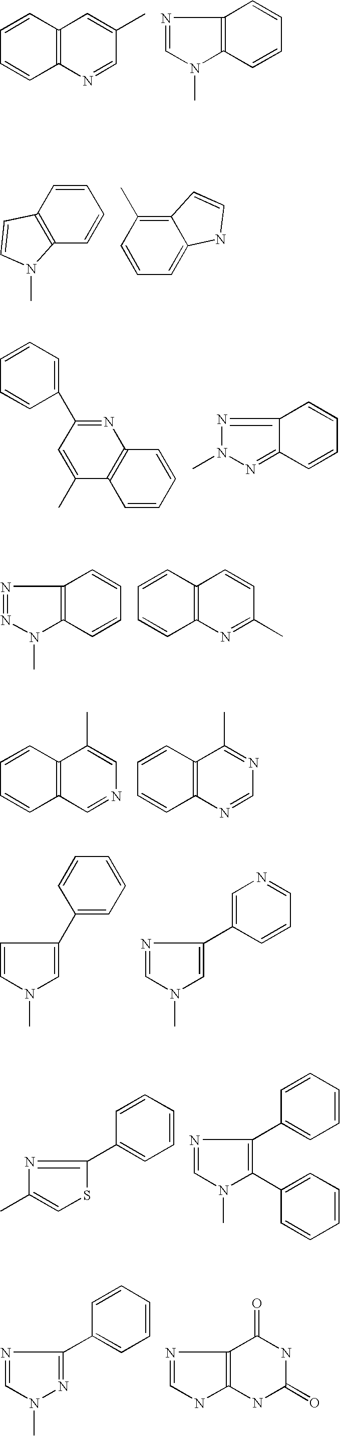

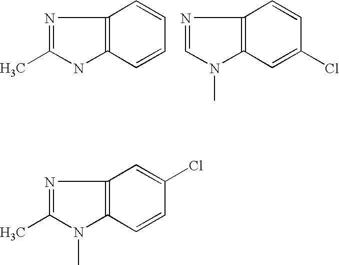

and the heterocyclic radicals envisaged in European Patent Applications 487-411, 596802, 676409 and 680967. These preferred heterocyclic radicals can be substituted by one or more functional groups.

Hal preferably represents a fluorine, chlorine or bromine atom.

Among the addition salts with acids which may be mentioned are the salts formed with acetic acid, propionic acid, trifluoroacetic acid, malic acid; tartaric acid, methanesulphonic acid, benzenesulphonic acid, p-toluenesulphonic acid and, especially, stearic acid, ethylsuccinic acid or laurylsulphonic acid.

The aryl radical is preferably a heterocyclic aryl radical. Among the preferred ketolides which may be mentioned are the compounds in which Ar represents a radical

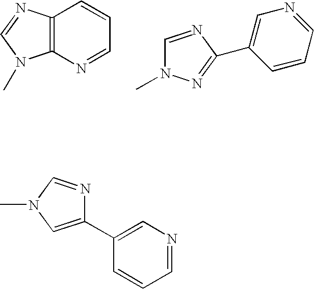

Among the preferred compounds of the invention which may be mentioned are the compounds of formula (I) whose names are given below: 11,12-dideoxy-3-de[(2,6-dideoxy-3-C-methyl-3-O-methyl-alpha-L-ribohexopyranosyl)oxy]-6-O-methyl-3-oxo-12,11-([oxycarbonyl [[[2-[4-(3-pyridyl)-1H-imidazol-1-yl]-ethoxy]methyl]iminol]erythromycin (compound P) described in Patent Application WO 98/25942 in Example 2 or 11,12-dideoxy-3-de((2,6-dideoxy-3-C-methyl-3-O-methyl-alpha-L-ribohexopyranosyl)oxy)-6-O-methyl-3-oxo-12,11-(oxycarbonyl((4-(3-(3-pyridyl)-1H-1,2,4-triazol-1-yl)butyl)imino)erythromycin (compound P₁) described in Patent EP 680967 in Example 35, or 11,12-dideoxy-3-de((2,6-dideoxy-3-C-methyl-3-O-methyl-alpha-L-ribohexopyranosyl)oxy)-2-fluoro-6-O-methyl-3-oxo-12,11-(oxycarbonyl((4-(4-(3-pyridyl)-1H-imidazol-1-yl)butyl)-imino))erythromycin (A isomer) (compound P₂) described in Patent EP 799833 in Example 3, or 11,12-dideoxy-3-de((2,6-dideoxy-3-C-methyl-3-O-methyl-alpha-L-ribohexopyranosyl)oxy)-6-O-methyl-3-oxo-12,11-(oxycarbonyl((4-(4-(3-pyridyl)-1H-imidazol-1-yl)butyl)imino))erythromycin (compound P₃) described in Patent EP 680967 in Example 34.

Among the ketolides which are particularly advantageous, mention may be made of the products in European Patents 676409, 680967 and 799833.

The ketolides exhibit an anti-platelet-aggregating and antithrombotic activity, as shown by the results obtained in the experimental section disclosed below.

The invention thus relates to pharmaceutical compositions intended for preventing arterial complications, such as cerebrovascular accidents, myocardial infarction and unstable angina following atherosclerosis.

The infectious agent Clamydia pneumoniae appears to play a role in the development of atherosclerosis in man.

The ketolides are active against Clamydia pneumoniae.

As a result, the anti-infectious properties against Clamydia pneumoniae which are associated with their anti-platelet-aggregating activity allow them to be used to combat the development of atherosclerosis and thrombotic complications.

The invention also relates to pharmaceutical compositions containing a ketolide defined above which are intended for preventing arterial thrombotic complications associated with atherosclerosis.

These compositions can be administered orally, rectally, parenterally or locally by topical application to the skin and mucous membranes, but the route of administration is the oral route.

They can be solid or liquid and can be in the pharmaceutical forms commonly used in human medicine, such as, for example, simple or sugar-coated tablets, gel capsules, granules, suppositories, injectable preparations, ointments, creams or gels; they are prepared by the usual methods. The active principle(s) may be incorporated therein with excipients usually used in these pharmaceutical compositions, such as talc, gum arabic, lactose, starch, magnesium stearate, cocoa butter, aqueous or non-aqueous vehicles, fatty sub-stances of animal or plant origin, paraffinic derivatives, glycols, various wetting, dispersing or emulsifying agents and preserving agents.

These compositions can also be in the form of a powder intended to be dissolved in a suitable vehicle, for example apyrogenic sterile water, at the time of use.

The dose administered is variable depending on the infection treated, the individual concerned, the route of administration and the product concerned. It can be, for example, between 50 and 600 mg per day via the oral route in an adult for the product P, P₁, P₂ or P₃.

### PHARMACOLOGICAL STUDY

### In Vitro Platelet Aggregation.

### Principle

The platelet aggregation is measured by the turbidimetry method inspired by Born [1], by detecting the optical transmission through a platelet-rich plasma (PRP) to which and aggregating agent has been added. When the platelets aggregate, the plasma becomes clear and the optical transmission increases.

### Preparation of the Platelet-Rich Plasma

Blood is taken (3 tubes per rabbit) by cardiac puncture from a rabbit into tubes containing sodium citrate. To obtain the platelet-rich plasma (PRP), the tubes are centrifuged at 160 g for 10 minutes. The supernatants are collected (PRP) and the pellet is re-centrifuged at 2000 g for 15 minutes to obtain the platelet-poor plasma (PPP). By dilution with the PPP, the PRP is adjusted to a concentration of 300,000 platelets per mm³±10%. The counting is carried out using a Coulter ZM counter.

### Aggregation

Tubes containing 320 μl of PRP are incubated at +37° C. for 30 minutes in pre-incubation wells.

The aggregometer is calibrated with the PPP for an optical transmission of 100% corresponding to a complete aggregation, and with the PRP obtained from the same rabbit for an optical transmission of 0% corresponding to the absence of aggregation.

The test product P is added in a volume of 40 μl. After incubation for 2 minutes, the aggregating agent (10 μM ADP, 0.2 mM sodium arachidonate or collagen 20% g/ml) is added in a volume of 40 μl. The aggregation begins immediately and can be seen on the printer.

On the plot obtained, the height of the aggregation curve is measured in cm from the baseline before addition of the aggregating agent, and then converted into mVolts (=1/OD) using the formula 10 mV=2.5 cm.

[1]—Born G.V.R., Aggregation of blood platelets by adenosine diphosphate and its reversal, Nature, 1962, 194, 927.

The results obtained are as follows:

Effect of Product P on In Vitro Platelet Aggregation—Comparison with Aspirin.

% of inhibition of the aggregation induced by
arachidonic acid⁺

| Concentrations | | Product P* | Aspirin** |
|---|---|---|---|
| 10⁻⁷ | M | 7 | — |
| 10⁻⁶ | M | 42 | 8 |
| 10⁻⁵ | M | 73 | 13 |
| 5 × 10⁻⁵ | M | — | 85 |
| 10⁻⁴ | M | 90 | 100 |

⁺The rabbit platelets are placed in the presence of the product at different concentrations and the arachidonic acid is then added in a concentration of 0.2 mM.
*n = 2 rabbits
**n = 4 rabbits except for the concentration 5 × 10⁻⁵ M, where n = 2.

The preferred products P₁, P₂ and P₃ mentioned above also show good activity on this in vitro platelet aggregation test.

What is claimed is:

1. A method of inhibiting platelet aggregation in a patient comprising administering to a patient in need thereof an effective amount of a ketolide, or its non-toxic pharmaceutically acceptable acid addition salt, sufficient to inhibit platelet aggregation in said patient.

2. The method of claim 1 wherein the ketolide has the formula 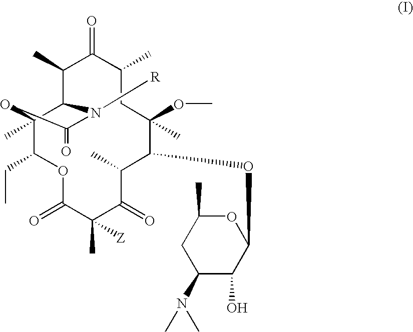
wherein R is —(CH₂)ₘOₙ(X)Y Ar, m and n are individually 0 or 1, X is selected from the group consisting of —(NH)ₐ—, —CH₂— and —SO₂—, a is 0 or 1, Y is —(CH₂)_{b}—(CH═CH)_{c}—(CH₂)_{d}, c is 0 or 1, b+c+d≦8, Z is hydrogen or halogen and Ar is unsubstituted or substituted aryl or heteroaryl.

3. The method of claim 1 wherein the ketolide is orally administered at from 50 to 500 mg per day.

4. The method of claim 1, wherein the ketolide is 11,12-dideoxy-3-de[(2,6-dideoxy-3-C-methyl-3-O-methyl-alpha-L-ribohexopyranosyl)oxy]-6-O-methyl-3-oxo-12,11-([oxycarbonyl[[[2-[4-(3-pyridinyl)-1H-imidazol-1-yl]ethoxy]-methyl]imino]]erythromycin.

5. The method of claim 1, wherein the ketolide is 11,12-dideoxy-3-de((2,6-dideoxy-3-C-methyl-3-O-methyl-alpha-L-ribohexopyranosyl)oxy)-6-O-methyl-3-oxo-12,11-(oxycarbonyl((4-(3-(3-pyridinyl)-1H-1,2,4-triazol-1-yl)butyl)imino)erythromycin.

6. The method of claim 1, wherein the ketolide is 11,12-dideoxy-3-de((2,6-dideoxy-3-C-methyl-3-O-methyl-alpha-L-ribohexopyranosyl)oxy)-2-fluoro-6-O-methyl-3-oxo-12,11-(oxycarbonyl((4-(4-(3-pyridinyl)-1H-imidazol-1-yl)butyl)imino))erythromycin (A isomer).

7. The method of claim 1, wherein the ketolide is 11,12-dideoxy-3-de((2,6-dideoxy-3-C-methyl-3-O-methyl-alpha-L-ribohexopyranosyl)oxy)-6-O-methyl-3-oxo-12,11-(oxycarbonyl((4-(4-(3-pyridinyl)-1H-imidazol-1-yl)butyl)imino))erythromycin.
